(19) United States
(12) Patent Application Publication
(10) Pub. No.: US 2006/0009857 A1
(43) Pub. Date: Jan. 12, 2006

(54) Method and apparatus for surface hardening implants

(76) Inventors: Phillip M. Gibbs, Winona Lake, IN (US); David W. Schroeder, Winona Lake, IN (US); Ryan C. Lakin, Warsaw, IN (US)

Correspondence Address: HARNESS, DICKEY & PIERCE, P.L.C., P.O. BOX 828, BLOOMFIELD HILLS, MI, 48303, US

(21) Appl. No.: 10/887,209

(22) Filed: Jul. 8, 2004

(51) Int. Cl. A61F 2/30; A61F 2/32; A61F 2/36

(52) U.S. Cl. 623/23.4; 623/22.2; 623/22.42; 623/22.43; 623/23.5

(57) ABSTRACT

A method for producing a prosthetic having metal articulating surfaces is disclosed. The method includes impinging at least a portion of the articulating surface with high energy laser electromagnetic radiation, to form a lasershot peened surface. A hardened portion of the surface is then post-processed to form a layer having a predetermined finish.

### FIELD OF THE INVENTION

The present invention relates to orthopedic implants and particularly to a method of surface hardening of bearing surfaces of the implant.

### BACKGROUND OF THE INVENTION

It is generally known in the art to use prosthetic devices to replace portions of the human anatomy that have been damaged due to injury or age. Often these prosthetic devices are formed of materials that are inherently strong yet easily formable. Many modular prosthetic devices are formed of at least metal stem portions that are inserted into long bones to provide a base for an external portion that extends from the boney portion. A taper or neck often interconnects the portion that extends from the bone, such as a head of a humerus or a femur, and the stem that is inserted in the bone. A taper may also be used to interconnect modular positions that are disposed within the bone after implantation. It is also known to provide bearing surfaces that must interact with one another while not wearing quickly or producing much wear debris.

The taper or neck that interconnects the two portions of the prosthesis, sometimes referred to as a Morse taper, must be strong enough to withstand cyclic loads that will be seen in a wide variety of anatomies, patient activity levels, and compromised boney constructs. The neck must also allow a range of movement that closely simulates the natural human anatomy. Other types of prosthetic devices are also modular and are formed from multiple interconnecting components. These components may also be interconnected by way of a Morse taper.

While materials generally used in these devices are inherently strong and have high tensile strengths, they require a particular thickness or mass to provide enough support for the portion of the anatomy that is being replaced. Due to this, the Morse taper is often larger and does not provide a full or natural range of motion. If the taper is for internal bone connection, a strong enough connection may produce a taper that is too big to fit into smaller bones. Due to this, it is desirable to produce prosthetic devices that include neck or interconnection portions that are small enough to fit into smaller bones and allow a full range of motion while being strong enough to support the stresses which the prosthetic will encounter.

One solution has been to provide new metal alloys that are particularly strong. These metal alloys may be formed into a myriad of shapes while still providing much of the support necessary for the prosthetic device. These new metal alloys, however, are still required to have large enough interconnection portions to provide the necessary strength to the materials.

Other known methods include the cold working or work hardening of prosthetics, such as that disclosed in the commonly assigned U.S. Pat. No. 6,067,701 entitled “Method for Forming a Work Hardened Modular Component Connector,” which is hereby incorporated by reference.

Cold working induces residual stresses within the component and may also produce the required or different residual stresses within the component. Particular residual stresses can be either compressive or tensile depending upon their nature. Compressive residual stresses are particularly desired. In particular, compressive residual stresses inhibit or stop cracks which may form in the prosthetic device. Furthermore, compressive stresses inhibit the initiation of a crack within the area which is loaded by external forces.

Compressive stress, especially near the surface of the component, also provides additional benefits. In particular, compressive stress near the surface can decrease fatigue and stress corrosion failures. In particular, these fatigue and stress corrosion failures originate at the surface and the compressive stress help inhibit such failures. In addition, the compressive residual stresses increase resistance to other undesired events such as fatigue failures, corrosion failure, stress corrosion cracking, hydrogen assisted cracking, fretting, galling, and corrosion caused by cavitation. Additionally, work hardening, which produces the compressive stresses, increases intergranular corrosion resistance, surface texturing, and closing of surface porosity.

Although compressive stresses, or other particular residual stresses, provide these many benefits, it is more beneficial to precisely create the desired residual stresses within the prosthetic device. Although exploratory cold working a component may produce the desired residual stresses, predetermining and work hardening components to produce predetermined residual stresses is preferable. Therefore, it is desired to provide a known process to produce within the prosthetic device, known and predetermined residual stresses that will provide compressive and tensile stresses, that are desired in a component.

Thus, it is desirable to have a method of producing prosthetic devices that leave no uncertainty to the strength being introduced into the prosthetic device. This would allow for more efficient manufacturing and an increase in prosthetic strength that survive the testing phase. That is, it is desirable to produce a prosthetic device that needs not be tested as often while still assuring that the prosthetic device will be able to handle the loads after being implanted.

### SUMMARY OF THE INVENTION

A method for producing a prosthetic having metal articulating surfaces is disclosed. The method includes impinging at least a portion of the articulating surface with high energy laser electromagnetic radiation, and allowing the surface to cool to form a layer having a predetermined hardness. A hardened portion of the surface is then post-processed to form a layer having a predetermined finish. In another embodiment of the present invention, a method for producing an acetabular cup is disclosed. The process subjects the hemispherical articulating surface of the acetabular cup to lasershot peening to form a surface having a predetermined hardness. Honing is performed on the surface to provide an articulating surface having a predetermined hardness and surface finish.

In another embodiment to the present invention, a implantable femoral prosthetic is provided having an articulating head portion. The surface of the head portion is subjected to lasershot peening to form a hardened surface. As with the previously described bearing, the head surface is machined to provide a articulating surface having a suitable surface finish.

In another embodiment to the present invention, a method of producing a Morse taper joint for a prosthetic is disclosed. At least one surface of the Morse taper is subjected to pulses of high intensity laser electromagnetic radiation to cause surface hardening of the Morse taper joint. Post-processing is conducted on the treated surface to produce the proper surface finish.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 represents an articulating acetabular prosthetic having a hardened surface;

FIG. 2 represents a cross-sectional view of a prosthetic assembly utilizing the prosthetic shown in FIG. 1;

FIGS. 3 and 4 represents the hardening of an articulating surface of the prosthetic shown in FIG. 1; and

FIGS. 5 and 6 represent the hardening of various surface of a femoral prosthetic.

### DETAILED DESCRIPTION OF THE PREFERRED EMBODIMENTS

The following description of the embodiments are merely exemplary in nature and are in no way intended to limit the invention, its application, or uses.

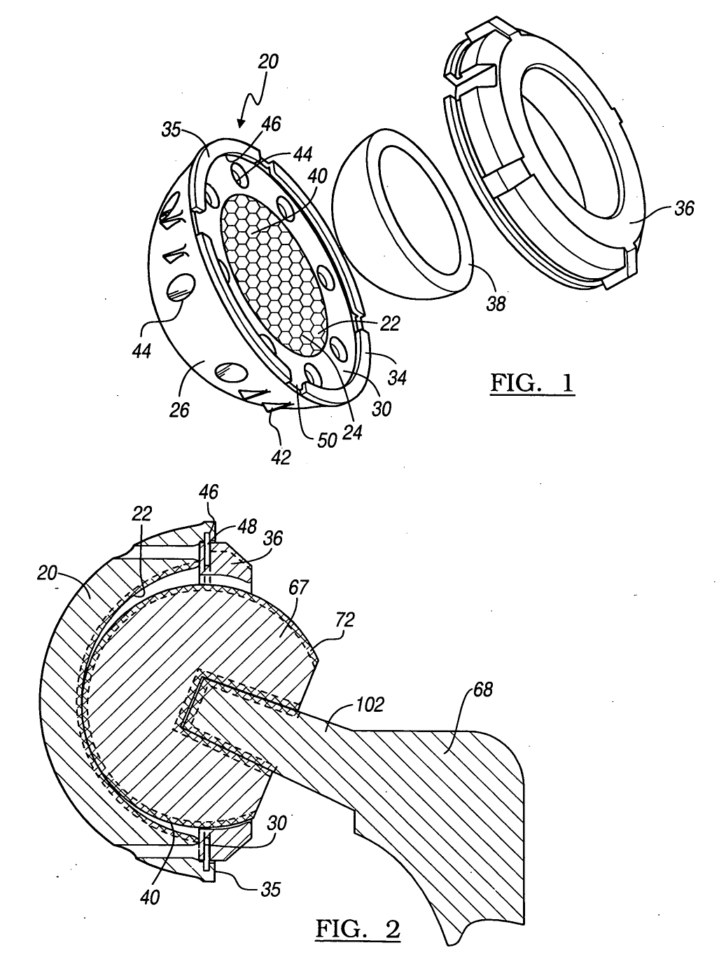

Referring to FIG. 1, an acetabular prosthesis 20 according to the teachings of a first embodiment of the present invention is shown. The acetabular prosthesis 20 has a inner surface 22 defining a first metal bearing surface 24 and an outer surface 26 capable of being coupled or impacted into a prepared acetabulum. As further descried below, the first metal bearing surface is hardened using a laser peening process. Defined between the outer surface 26 and the inner surface 22 is a peripheral surface or rim 30. Disposed on the peripheral surface 30 is a locking mechanism 34. The locking mechanism 34 is capable of coupling a second prosthetic such as an optional constraining ring 36, or an optional insert bearing 38 into a bearing cavity 40 which is defined by the inner surface 22 of the acetabular prosthesis 20. The optional bearing inert 38 can be biocompatible polymer, ceramic or hardened metal. The second prosthetic substantially surrounds a head of a femoral component (as further described later), were substantially encloses includes for example a slotted constraining ring.

The outer surface 26 of the acetabular prosthesis 20 defines a plurality of locking projections 42 for coupling the acetabular prosthesis 20 to the prepared acetabulum. It is envisioned that the outer surface 26 can be surface treated to facilitate bone ingrowth or fixation to bone cement, such as by porous coating.

FIG. 1 further illustrates that the acetabular prosthesis 20 can have a plurality of through holes 44 to assist in fixation to the prepared acetabulum. Defined in the peripheral surface 30 is the plurality of through holes 44 for mounting the acetabular prosthesis 20 to the prepared acetabulum 28 using standard bone coupling fasteners or screws.

Although there are many ways to couple the constraining ring 36 or insert bearing 38 into the bearing cavity 40 defined by the inner surface 22 such as fasteners or tabs, shown is the locking mechanism 34 is formed by a locking flange 35 which defines a coupling groove 46. The coupling groove 46 is designed to accept the locking ring 48. The locking flange 35 has a plurality of alignment notches 50 disposed therein to facilitate the acceptance of the locking ring 48 and alignment of the acetabular prosthesis 20, further discussed herein.

FIGS. 2 illustrates a cross-sectional view of head 67 of a femoral prosthesis 68 into the acetabular prosthesis 20, along with the use of the optional constraining ring 36. It should again be noted that as the inner surface 22 of the acetabular prosthesis 20 is a highly polished hardened metal bearing surface formed from a bio-compatible material such as titanium, cobalt chrome, stainless steel, etc. The first head 67 of the femoral prosthesis 68 will articulate within the bearing cavity 40 defined by the inner surface 22 of the acetabular prosthesis 20. As can be seen, the first head 67 is inserted into the bearing cavity 40. Next, the constraining ring 36, which was previously disposed about the neck 102 of the femoral implant 68, is positioned adjacent to the-peripheral surface 30 of the acetabular prosthesis 20.

The locking ring 48 is inserted into the coupling groove 46 defined by the locking flange 35 and the constraining ring 36 is released to affix the constraining ring 36 onto the acetabular prosthesis 20. In this way, a metal-metal articulating bearing surface is formed between the inner surface 22 and the femoral head 67. As is shown, the first femoral head 67 engages the metal bearing surface 24. The locking ring 48 is positioned within the constraining ring groove 56 to fix the constraining ring 36 to the acetabular prosthesis 20, thus locking the first head 67 into its proper orientation. FIG. 2 depicts the location of the locking ring 48 with respect to the acetabular prosthesis 20 and constraining ring 36. Further shown is the location of the fastener or screw to the locking mechanism 34.

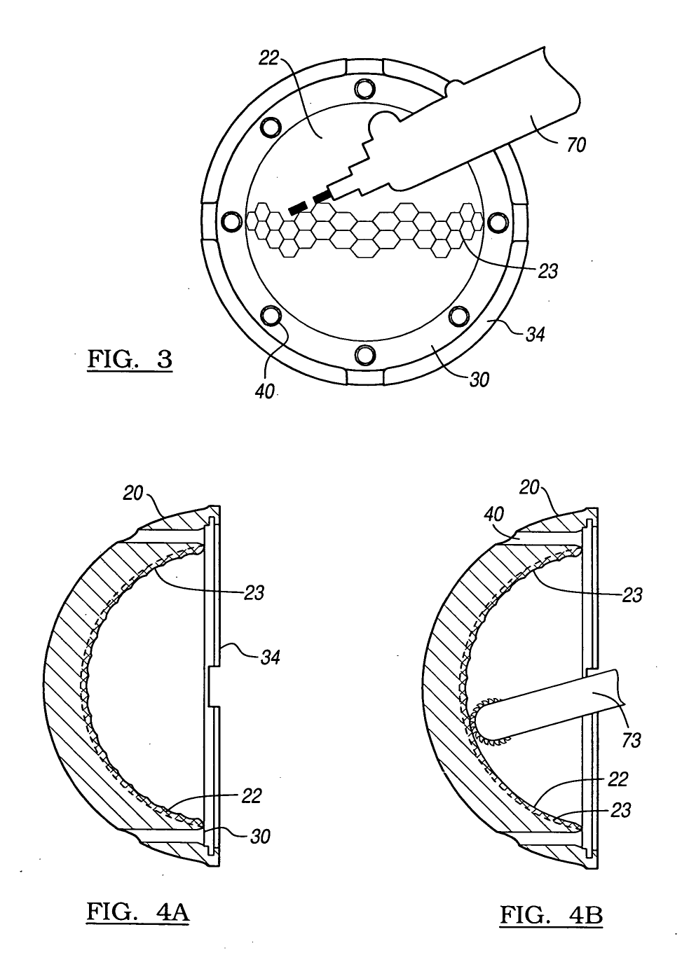

As shown in FIG. 3, to counter fatigue failure of portions of the bearing elements from deformities that can develop in the bearing articulating surfaces 22, the present invention provides a laser shock peened curved surface 23 on at least a portion of the contact surfaces of the bearing elements. The pre-stressed laser shock peened curved regions 23 have deep compressive residual stresses which are imparted by laser shock peening. The compressive residual stresses extend into the bearing elements from the laser shock peened surfaces 23. Preferably, the entire curved spherical surface of the femoral head 67 are so laser shock peened as are the curved contact semi-spherical outer and inner circumferential surfaces of the acetabular and femoral bearing surfaces, respectively.

The laser beam shock induced deep compressive residual stresses in the compressive pre-stressed regions are generally about 50-150 KPSI (Kilo Pounds per Square Inch). These pre-stressed regions extend from the laser shock surfaces to a depth of about 20-50 mils into laser shock induced compressive residually pre-stressed regions. The laser beam shock induced deep compressive residual stresses are produced by repetitively firing a high energy laser beam from the laser 70 that is focused on surface which is covered with paint to create peak power densities having an order of magnitude of a gigawatt/cm². As is known, the laser beam is fired through a curtain of flowing water that is flowed over the surface and the surface is ablated generating plasma which results in shock waves on the surface of the material. These shock waves are re-directed towards the surface by the curtain of flowing water to generate traveling shock waves (pressure waves) in the material below the surface. The amplitude and quantity of these shock waves determine the depth and intensity of the compressive stresses. The surface is used to protect the target surface and also to generate plasma. Ablated surface material is washed out by the curtain of flowing water.

FIGS. 4a and 4b show the hardening of the acetabular component 20. The laser peened surface 23 has a surface roughness which would prevent its use as an orthopedic implant bearing. As such, further surface honing and polishing of the bearing surface using a honing instrument 73 provides a prosthetic having a bearing surface with a predetermined hardness and surface finish.

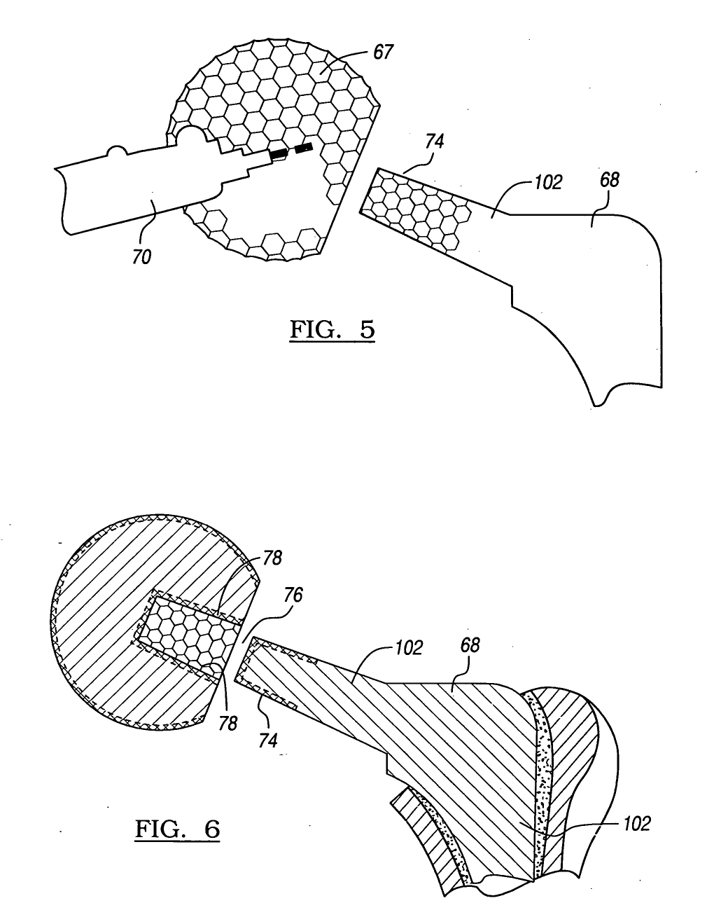

FIGS. 5 and 6 represents the hardening of surfaces of the femoral prosthetic. As seen in FIG. 5, the exterior surface 72 of the articulating head and Morse taper is hardened as previously described by impinging the surface with a high intensity laser. The stem portion 102 of the femoral component 68, which is configured to be coupled to a bone, has a tapered neck with a male portion 74 of a Morse taper joint 76. A portion of a coupling surface of the male portion of the Morse taper joint is hardened using overlapping spots of laser shock peening which induces residual stress that extends into the prosthetic from the laser shock peened surface. FIG. 6 represents a cross-sectional view of the machined femoral prosthetic shown in FIG. 5. Shown is the female portion 78 of the Morse taper 76 which has a coupling surface 78 which has been hardened.

In general, a method for implanting a medical device, for example a femoral prosthetic 68, into a patient is described. Prior to implantation, at least one surface of a femoral prosthetic 68 is subjected to laser shock peening. The surface can be an internal coupling surface such as a Morse taper joint or can be the bearing surface of the femoral head. The femoral prosthetic 68, which can be a single piece or a modular component, is then implanted into a prepared joint. In this regard, the stem of the femoral prosthetic 68 is inserted into a resected femur. In the case of a modular femoral prosthetic 68, the surface hardened head 67 is coupled to the stem portion 102 of the femoral component 68.

The description of the invention is merely exemplary in nature and, thus, variations that do not depart from the gist of the invention are intended to be within the scope of the invention. For example, the teachings of the present invention are disclosed in a description of an acetabular and femoral prosthetic. It is however envisioned that the teaching are equally applicable to other joints, for example knee, ankle, or shoulder joints. Such variations are not to be regarded as a departure from the spirit and scope of the invention.

What is claimed is:

1. A prosthetic for implantation in a patient formed of a biocompatible material comprising:
an engagement surface;
a first bearing surface, a portion of said bearing surface being a laser shock peened surface,
wherein a layer of the biocompatible material includes a compressive residual stress imparted by laser shock peening that extends into the prosthetic from the laser shock peened surface.

2. The prosthetic according to claim 1 wherein the first bearing surface is spherical.

3. The prosthetic according to claim 1 wherein the first bearing surface is convex.

4. The prosthetic according to claim 1 wherein the first bearing surface is concave.

5. The prosthetic according to claim 1 further comprising a locking mechanism configured to couple a second prosthetic implant having a second bearing surface, the first bearing surface being configured to substantially surround an articulating head of a third prosthetic.

6. The prosthetic according to claim 5 wherein said second prosthetic is selected from a group of a constraining ring, a slotted constraining ring, a bearing insert, and a bearing insert having an integral constraining ring and combinations thereof.

7. The prosthetic according to claim 1 wherein the first bearing surface is formed of a biocompatible metal.

8. A prosthetic for implantation in a patient formed of a biocompatible material:
a first member having a bone engagement surface and a first contact surface;
a second member, defining a second contact surface which is configured to contact said first contact surface when the prosthetic is implanted in the patient; and
wherein a portion of one of the first or second contact surface is a laser shock peened surface.

9. The prosthetic according to claim 8 wherein said first contact surface is a hemispherical bearing surface.

10. The prosthetic according to claim 8 wherein one of said first and second members further defines a generally spherical bearing surfaces.

11. The prosthetic according to claim 8 wherein the first and second contact surfaces are coupling surfaces of a Morse taper joint.

12. A prosthetic comprising:
a first portion defining a male portion of a Morse taper joint having a first coupling surface;
a second portion defining a female portion of a Morse taper joint having a second coupling surface; and
wherein at least one of the first and second coupling surfaces is a first laser shock peened surface and wherein a compressive residual stress is imparted by the laser shock peening which extends into the prosthetic from the first laser shock peened surface.

13. The prosthetic according to claim 12 wherein both the first and second coupling surfaces are laser peened surfaces.

14. The prosthetic according to claim 12 wherein the first portion comprises a bone engaging surface.

15. The prosthetic according to claim 14 wherein the second portion comprises an articulating surface.

16. The prosthetic according to claim 15 wherein the articulating surface is a second laser peened surface.

17. The prosthetic according to claim 14 further comprising a first bearing surface, a portion of said bearing surface being a laser shock peened surface, and
a layer of material having a compressive residual stress imparted by laser shock peening extends into the prosthetic from the laser shock peened surface.

18. A method for implanting a medical device comprising:
subjecting a surface of a first prosthetic to laser shock peening; and
implanting the first prosthetic into a prepared joint.

19. The method according to claim 18, further including coupling a femoral prosthetic within the surface of the first prosthetic.

20. The method according to claim 18, wherein the surface is a concave bearing surface and wherein the first prosthetic comprises a locking mechanism which is configured to fixably accept a second prosthetic having a second bearing surface.

21. The method according to claim 20, wherein the first prosthetic is an acetabular cup and the second prosthetic is a constraining ring.

22. The method according to claim 20, wherein the surface is a bearing surface.

23. The method according to claim 20, wherein the surface is a coupling surface of a Morse taper joint.

24. The method according to claim 18 further comprising having a portion of the surface which was subjected to laser shock peening.
